(19) United States
(12) Patent Application Publication
(10) Pub. No.: US 2016/0220471 A1
(43) Pub. Date: Aug. 4, 2016

(54) PROCESS FOR TREATING KERATIN FIBRES WITH A POLYSACCHARIDE AND A SILANE ADDED TO THE WATER

(71) Applicant: L'OREAL, Paris (FR)

(72) Inventors: Nawel BAGHDADLI, Massy (FR); Isabelle PASINI, Aulnay-sous-Bois (FR)

(21) Appl. No.: 15/021,418

(22) PCT Filed: Sep. 11, 2014
(86) PCT No.: PCT/EP2014/069380

§ 371 Date: Mar. 11, 2016

(30) Foreign Application Priority Data
Sep. 12, 2013 (FR) 1358762

(51) Int. Cl. A61K 8/73; A61Q 5/12; A45D 2/00; A61K 8/58

(52) U.S. Cl. CPC A61K 8/73; A61K 8/585; A61Q 5/12; A45D 2/001; A61K 2800/884

(57) ABSTRACT

The invention relates to a process for treating keratin fibres, in particular human keratin fibres such as the hair, comprising: (i) a step of applying an oxidized polysaccharide, (ii) a step of heating to a temperature of at least 100° C., (iii) a step of applying a silane added to the water. The process makes it possible to obtain good hair-conditioning cosmetic properties, with a long-lasting effect. The invention also relates to a cosmetic composition and a kit that are used for performing the treatment process.

The invention relates to a cosmetic process for treating keratin fibres, in particular human keratin fibres such as the hair, using an oxidized polysaccharide combined with a silane added to the water.

Hair is generally damaged and embrittled by the action of external atmospheric agents such as light, sunlight and bad weather, and also by mechanical or chemical treatments, such as brushing, combing, dyeing, bleaching, permanent-waving, relaxing and repeated washing. Hair is thus damaged by these various factors and may in the long run become dry, coarse, brittle or dull or split or limp.

Thus, to overcome these drawbacks, it is common practice to resort to hair treatments which use compositions intended for conditioning the hair appropriately by giving it satisfactory cosmetic properties, especially a soft feel (the hair is no longer coarse), good disentangling properties leading to easy combing, and good manageability of the hair which is thus easy to shape.

These haircare compositions may be, for example, conditioning shampoos, hair conditioners, masks or sera.

However, the conditioning effect obtained fades out in the course of successive shampoo washes and does not show satisfactory persistence on shampooing.

In the field of dyeing, patent application FR 2 944 967 discloses the use of oxidized polysaccharides for protecting the colour of keratin fibres that have been artificially dyed, especially by oxidation dyeing or direct dyeing.

There is thus a need for a process for treating keratin fibres, in particular the hair, that is capable of durably conditioning the keratin fibres, the conditioning effect being persistent after one or more shampoo washes performed on the treated keratin fibres.

The Applicant has discovered that the application to keratin fibres, in particular the hair, of oxidized polysaccharide and of a silane added to the water as defined below followed by a heating step makes it possible to obtain good hair-conditioning cosmetic properties, with a durable effect over time, especially after one or more shampoo washes.

Thus, one subject of the invention is a process for treating keratin fibres, in particular the hair, comprising:

(i) a step of applying to the keratin fibres at least one oxidized polysaccharide as defined below;

(ii) a step of heating the keratin fibres to a temperature of at least 100° C., preferably ranging from 100 to 250° C.,

(iii) a step consisting in applying to the said fibres an aqueous cosmetic composition to which is added a silane as defined below.

The treatment process according to the invention makes it possible to obtain good keratin fibre-conditioning cosmetic properties.

In particular, hair treated via the process according to the invention remains managed since no presence of frizziness is observed. Thus, the hairs are aligned, smooth and disentangle easily, which makes them easier to comb. The treated hair also has more body (it is not limp) and is thus easier to style. The treated hair is well shaped.

Moreover, the treated hair is also shinier and has a softer feel. It is stronger and less brittle.

After treatment, the hair is not lank, and has a natural feel.

The process according to the invention has the advantage of giving good persistence of these good hair-conditioning cosmetic properties after shampooing. Thus, the treated hair is durably conditioned.

The process according to the invention also has the advantage of not bringing about a change in the colour of the treated hair.

A subject of the invention is also a cosmetic composition comprising, in a physiologically acceptable aqueous medium, a polysaccharide and a silane, added to the composition, of formula (II) as defined below.

A subject of the invention is also a kit comprising:

a first cosmetic composition comprising an oxidized polysaccharide as defined below and a second aqueous cosmetic composition comprising a silane, added to the composition, of formula (II) as defined below, the first and second compositions each being packaged in a separate packaging assembly.

A subject of the invention is also a kit comprising:

either a cosmetic composition comprising, in an aqueous medium, an oxidized polysaccharide and a silane, added to the composition, of formula (II) as defined below, the composition being contained in a packaging assembly,

or a first cosmetic composition comprising an oxidized polysaccharide as defined below and a second aqueous cosmetic composition comprising a silane, added to the composition, of formula (II) as defined below, the first and second compositions each being packaged in a separate packaging assembly,

and a device for heating the keratin fibres to a temperature of at least 100° C., preferably ranging from 100 to 250° C., such as those described below.

The composition packaging assembly is, in a known manner, any packaging that is suitable for storing cosmetic compositions (especially cans, tube, spray can or aerosol can).

Such a kit makes it possible to perform the keratin fibre treatment process according to the invention.

The oxidized polysaccharide(s) used in the process according to the invention are preferably anionic or nonionic polysaccharides.

The anionic or nonionic oxidized polysaccharides consist of monosaccharide units that may comprise five or more carbon atoms, preferably six or more carbon atoms, and more particularly six carbon atoms.

The nonionic or anionic oxidized polysaccharides comprise one or more aldehyde groups and optionally one or more anionic groups.

These anionic groups are preferably carboxyl or carboxylate groups.

The anionic or nonionic oxidized polysaccharides according to the invention may be represented by formula (I) below:

P—(CHO)ₘ(COOX)ₙ (I)

in which:

P represents a polysaccharide chain consisting of monosaccharides comprising 5 carbon atoms or more than 5 carbon atoms, preferably 6 or more than 6 carbon atoms and more particularly 6 carbon atoms;

X is chosen from a hydrogen atom, the ions derived from an alkali metal or an alkaline-earth metal such as sodium or potassium, ammonia, organic amines such as monoethanolamine, diethanolamine, triethanolamine and 3-amino-1,2propanediol and basic amino acids such as lysine, arginine, sarcosine, ornithine and citrulline,

m+n is greater than or equal to 1,

m is such that the degree of substitution of the polysaccharide with one or more aldehyde groups (DS(CHO)) is within the range from 0.001 to 2 and preferably from 0.005 to 1.5,

n is such that the degree of substitution of the polysaccharide with one or more carboxylic groups (DS(COOX)) is within the range from 0 to 2 and preferably from 0.001 to 1.5.

The term “degree of substitution DS(CHO) or DS (COOX) of the polysaccharides according to the invention” means the ratio between the number of carbons oxidized as an aldehyde or carboxylic group for all the repeating units and the number of elemental monosaccharides (even opened by preoxidation) constituting the polysaccharide.

The groups CHO and COOX may be obtained during the oxidation of certain carbon atoms, for example in position C₂, C₃ or C₆, of a saccharide unit comprising 6 carbon atoms. Preferably, the oxidation may take place at C₂ and at C₃, more particularly from 0.01% to 75% by number and preferably from 0.1% to 50% by number of the rings having possibly been opened.

The polysaccharide chain, represented by P, is preferably chosen from inulins, celluloses, starches, guar gums, xanthan gums, pullulan gums, alginate gums, agar-agar gums, carrageenan gums, gellan gums, gum arabics, xyloses and tragacanth gums, and derivatives thereof, cellobiose, maltodextrin, scleroglucan, chitosan, ulvan, fucoidan, alginate, pectin, heparin and hyaluronic acid, or mixtures thereof.

More preferentially, the polysaccharide chain is chosen from inulins and starches.

Even more preferentially, the polysaccharide chain is inulin.

The term “derivative” means the compounds obtained by chemical modification of the mentioned compounds. They may be esters, amides or ethers of the said compounds.

The oxidation may take place according to a process known in the art, for example according to the process described in FR 2 842 200, in document FR 2 854 161 or in the article “Hydrophobic films from maize bran hemicelluloses” by E. Fredon et al., Carbohydrate Polymers 49, 2002, pages 1 to 12. Another oxidation process is described in the article “Water-soluble oxidized starches by peroxide reaction extrusion” Industrial Crops and Products 75 (1997) 45-52—R. E. Wing, J. L. Willet. These oxidation processes are easy to perform, are efficient and do not generate any toxic by-products or by-products that are difficult to remove.

The peroxides that may be used in these oxidation processes may be an alkali metal or alkaline-earth metal percarbonate or perborate, an alkyl peroxide, peracetic acid or hydrogen peroxide. Hydrogen peroxide is particularly preferred, insofar as it is readily accessible and does not produce interfering by-products.

The amount of peroxide in the reaction medium is between 0.05 and 1 molar equivalent per glucose unit of the polysaccharide, preferably between 0.1 and 0.8 molar equivalent. It is preferable to add the peroxide in successive portions, leaving the reaction medium stirring between two additions.

A single phthalocyanin or a mixture of phthalocyanins, for example a mixture of cobalt phthalocyanin and of iron phthalocyanin, may be used as catalyst in the oxidation process. The amount of catalyst depends on the desired degree of substitution. In general, a small amount, for example an amount corresponding to 0.003 to 0.016 molar equivalent per 100 glucose units of polysaccharide, is suitable for use.

The process may also be performed by placing the polysaccharide in pulverulent form in contact with the catalyst dissolved in a small volume of water and with the peroxide. This process is referred to as a “semi-dry” process.

The process may be performed by reactive extrusion in the presence of peroxide.

More preferentially, the polysaccharide is obtained by oxidation of inulin, cellulose, carboxymethylcellulose, hydroxyethylcellulose, hydroxypropylcellulose, hydroxypropylmethylcellulose, methylcellulose, starch, starch acetate, hydroxyethyl starch, hydroxypropyl starch, guar gum, carboxymethyl guar gum, carboxymethylhydroxypropyl guar gum, hydroxyethyl guar gum, hydroxypropyl guar gum, xylose, xanthan gum or carrageenan gum, cellobiose, maltodextrin, scleroglucan, chitosan, ulvan, fucoidan, alginate, pectin, heparin and hyaluronic acid, or mixtures thereof.

Preferentially, the polysaccharide is obtained by oxidation of inulin or starch.

Preferentially, the polysaccharide is obtained by oxidation of inulin.

According to one embodiment, the polysaccharide is obtained by oxidation of inulin by performing a reactive extrusion process in the presence of hydrogen peroxide.

The polysaccharide chain before and after oxidation preferably has a weight-average molecular mass ranging from 400 to 15 000 000, better still from 500 to 10 000 000 and more particularly from 500 to 50 000 g/mol.

The polysaccharides that are most particularly preferred in the invention are those corresponding to formula (I) in which: P represents a polymer chain derived from inulin or from starch, m is such that the degree of substitution of the polysaccharide with one or more aldehyde groups (DS(CHO)) is within the range from 0.005 to 2.5, n is such that the degree of substitution of the polysaccharide with one or more carboxylic groups (DS(COOX)) is within the range from 0.001 to 2.

Even more preferably, P represents a polymer chain derived from inulin, m is such that the degree of substitution of the polysaccharide with one or more aldehyde groups (DS(CHO)) is within the range from 0.01 to 1, n is such that the degree of substitution of the polysaccharide with one or more carboxylic groups (DS(COOX)) is within the range from 0.01 to 2.

Advantageously, the step of applying the oxidized polysaccharide consists in applying a cosmetic composition comprising the oxidized polysaccharide especially in a content ranging from 0.05% to 15% by weight, preferably ranging from 0.1% to 10% by weight and more preferentially ranging from 0.2% to 6% by weight relative to the total weight of the composition.

The process according to the invention uses an aqueous cosmetic composition to which is added a silane of formula (II) as defined below. The silane (II) added to the aqueous composition, on contact with water, hydrolyses and condenses to form a polymer (or an oligomer). The composition may thus comprise a polymer that may be obtained by hydrolysis of silane (II) followed by condensation of the hydrolysed silane.

The silane used corresponds to formula (II) below:

R₁Si(OR₂)_{z}(R₃)ₓ (II)

in which:
R₁ is a linear or branched, saturated or unsaturated, cyclic or acyclic C₁-C₆ hydrocarbon-based chain substituted with a group chosen from the following groups:
   amine NH₂ or NHR with R=C₁-C₄ alkyl,
   an aryl or aryloxy group substituted with an amino group or with a C₁-C₄ aminoalkyl group;
   R₁ possibly being interrupted in its chain with a heteroatom (O, S, NH) or a carbonyl group (CO), R₁ being linked to the silicon atom directly via a carbon atom,
R₂ and R₃, which may be identical or different, represent a linear or branched alkyl group comprising from 1 to 6 carbon atoms,
z denotes an integer ranging from 1 to 3, and
x denotes an integer ranging from 0 to 2,

with z +x =3.

Preferably, R₂ represents an alkyl group comprising from 1 to 4 carbon atoms.

Preferably, R₂ represents a linear alkyl group comprising from 1 to 4 carbon atoms

Preferably, R₂ represents an ethyl group.

Preferably, R₃ represents an alkyl group comprising from 1 to 4 carbon atoms.

Preferably, R₃ represents a linear alkyl group comprising from 1 to 4 carbon atoms

Preferably, R₃ represents a methyl or ethyl group.

Preferably, R₁ is an acyclic chain.

Preferably, R₁ is a linear or branched, saturated or unsaturated C₁-C₆ hydrocarbon-based chain, substituted with an amine group NH₂ or NHR (R=C₁-C₆ alkyl, C₃-C₆ cycloalkyl or C₆ aromatic). Preferentially, R₁ is a saturated linear C₁-C₆ hydrocarbon-based chain substituted with an amine group NH₂. More preferentially, R₁ is a saturated linear C₂-C₄ hydrocarbon-based chain substituted with an amine group NH₂.

Preferably, R₁ is a saturated linear C₁-C₆ hydrocarbon-based chain substituted with an amine group NH₂.

R₂ represents an alkyl group comprising from 1 to 4 carbon atoms.

R₃ represents an alkyl group comprising from 1 to 4 carbon atoms.

Preferably, z is equal to 3.

Preferably, the silane of formula (II) is chosen from 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane and N-(2-aminoethylaminomethyl)phenethyltrimethoxysilane.

Preferably, the silane (II) is chosen from 3-aminopropyltriethoxysilane (APTES), 3-aminoethyltriethoxysilane (AETES), 3-aminopropylmethyldiethoxysilane and N-(2-aminoethyl)-3-aminopropyltriethoxysilane.

Preferably, the silane (II) is 3-aminopropyltriethoxysilane (APTES).

Advantageously, the aqueous cosmetic composition comprising the silane (II) added may be obtained by adding to the aqueous composition an amount of silane of formula (II) ranging from 0.1% to 15% by weight, preferably ranging from 0.5% to 10% by weight and preferentially ranging from 0.5% to 5% by weight, relative to the total weight of the composition.

According to one embodiment of the process according to the invention, the oxidized polysaccharide and the silane added to the water are present in separate cosmetic compositions. They are therefore applied separately to the keratin fibres. According to another embodiment of the process according to the invention, the oxidized polysaccharide and the silane added to the water are present in the same cosmetic composition. They are therefore applied simultaneously to the keratin fibres.

The cosmetic compositions used according to the invention contain a physiologically acceptable medium, i.e. a medium that is compatible with human keratin materials such as the skin (of the body, face, around the eyes or the scalp), the hair, the eyelashes, the eyebrows, bodily hair, the nails or the lips.

The physiologically acceptable medium of the composition(s) used in the process according to the invention is advantageously an aqueous medium. It may consist, for example, of water or of a mixture of water and of at least one cosmetically acceptable organic solvent. Examples of organic solvents that may be mentioned include C₂-C₄ lower alcohols, such as ethanol and isopropanol; polyols, especially those containing from 2 to 6 carbon atoms, for instance glycerol, propylene glycol, butylene glycol, pentylene glycol, hexylene glycol, dipropylene glycol or diethylene glycol; polyol ethers, for instance 2-butoxyethanol, propylene glycol monomethyl ether and diethylene glycol monomethyl ether or monoethyl ether; and mixtures thereof.

Preferably, the cosmetic composition comprises from 50% to 99.5% by weight of water relative to the weight of the composition.

The composition used according to the invention may also contain one or more cosmetic additives chosen from nonionic, anionic, cationic and amphoteric surfactants, vitamins and provitamins, including panthenol, sunscreens, fillers, dyestuffs, nacreous agents, opacifiers, sequestrants, film-forming polymers, plasticizers, thickeners, oils, antioxidants, antifoams, moisturizers, emollients, penetrants, fragrances and preserving agents.

The composition used according to the invention may be in any galenical form conventionally used for application to the hair and especially in the form of aqueous solutions, aqueous-alcoholic solutions, oil-in-water (O/W), water-in-oil (W/O) or multiple (triple: W/O/W or O/W/O) emulsions, aqueous gels or aqueous-alcoholic gels. These compositions are prepared according to the usual methods. Preferably, the composition is in the form of an aqueous or aqueous-alcoholic solution or gel.

The process according to the invention comprises a step of heating the keratin fibres to a temperature of at least 100° C., preferably ranging from 100 to 250° C. Preferably, the step of heating the keratin fibres is performed at a temperature ranging from 150 to 220° C., preferably ranging from 160° C. to 220° C., preferentially ranging from 160° C. to 200° C. and especially ranging from 170° C. to 190° C.

This heating step is advantageously performed using an iron.

The heating step is necessary to optimize the effects of the process.

For the purposes of the present invention, the term “iron” means a device for heating keratin fibres by placing the said fibres and the heating device in contact.

The end of the iron which comes into contact with the keratin fibres generally has two flat surfaces. These two surfaces may be made of metal or ceramic. In particular, these two surfaces may be smooth or crimped or curved.

The heating step may be performed by means of a straightening iron, a curling iron, a crimping iron or a steam iron. Preferably, the heating step is performed using a straightening iron.

As examples of irons that may be used in the straightening process according to the invention, mention may be made of any type of flat iron, and in particular, in a nonlimiting manner, those described in patents U.S. Pat. No. 5,957,140 and U.S. Pat. No. 5,046,516. The iron may be applied by successive separate strokes lasting a few seconds or by gradual movement or sliding along the locks of keratin fibres, especially of hair.

Preferably, the iron is applied in the process according to the invention by a continuous movement from the root to the end of the hair, in one or more passes, in particular in two to twenty passes. The duration of each pass of the iron may last from 2 seconds to 1 minute.

Preferably, the step of heating the keratin fibres is performed for a time that may range from 2 seconds to 30 minutes, preferentially from 2 seconds to 20 minutes, better still from 2 seconds to 10 minutes, better still from 2 seconds to 5 minutes and even better still from 2 seconds to 2 minutes.

The process according to the invention may also comprise an additional step of drying the keratin fibres after the application of the oxidized polysaccharide and/or of the silane added to the water or of the cosmetic composition(s) containing the same and before the step of heating the keratin fibres performed at a temperature of at least 100° C. The drying step may be performed using a hairdryer or a hood or by open drying. The drying step is advantageously performed at a temperature ranging from 20 to 70° C.

After the drying step, the keratin fibres may be optionally rinsed with water or washed with a shampoo. The keratin fibres are then optionally dried using a hairdryer or a hood or in the open air.

According to one embodiment, the process according to the invention is performed on natural keratin fibres, especially natural hair.

According to another embodiment, the process according to the invention is performed on damaged keratin fibres, especially hair. As indicated previously, the term “damaged hair” means dry or coarse or brittle or split or limp hair.

According to another embodiment, the treatment process according to the invention is preferably performed on sensitized keratin fibres, especially hair, such as bleached, artificially dyed, relaxed or permanent-waved fibres.

The process according to the invention may be performed on keratin fibres, especially hair, which is dry or wet. Preferentially, the process is performed on dry keratin fibres, especially dry hair.

After application to the keratin fibres of the oxidized polysaccharide and/or of the silane added to the water, or of a cosmetic composition containing the same, and before performing the step of heating the keratin fibres, the oxidized polysaccharide and/or the silane added to the water or the composition(s) containing the same may be applied for a time ranging from 1 to 60 minutes, preferably ranging from 2 to 50 minutes and preferentially ranging from 5 to 45 minutes. The composition may be left on at a temperature ranging from 15° C. to 45° C., preferably at room temperature (25° C.).

The cosmetic composition(s) described previously are advantageously applied to the keratin fibres in an amount ranging from 0.1 to 10 grams and preferably from 0.2 to 5 grams of composition per gram of keratin fibres.

After application of the cosmetic composition to the keratin fibres, they may be manually dried to remove the excess composition or washed with water or with a shampoo.

According to a first embodiment of the process according to the invention, the following steps are performed, in the following order: the step of applying the oxidized polysaccharide, then the step of applying the aqueous cosmetic composition comprising the addition of the silane and then the heating step. Advantageously, the polysaccharide is present in a first cosmetic composition and the silane added to the water is present in a second aqueous cosmetic composition. This second composition is separate from the first composition.

According to a second embodiment of the process according to the invention, the following steps are performed, in the following order: simultaneously, the step of applying the oxidized polysaccharide and the step of applying the aqueous cosmetic composition comprising the addition of the silane and then the heating step. Advantageously, the oxidized polysaccharide and the silane added to the water are present in a single aqueous cosmetic composition.

According to a third embodiment of the process according to the invention, the following steps are performed, in the following order: the step of applying the oxidized polysaccharide, then the heating step, then the step of applying the aqueous cosmetic composition comprising the addition of the silane and then optionally an additional heating step.

The treatment process according to the invention may be performed before, during and/or after an additional process of cosmetic treatment of the keratin fibres, such as a process for temporarily shaping (shaping with curlers, a crimping iron or a straightening iron) or a process for durably shaping (permanent-waving or relaxing) the keratin fibres.

The treatment process may be performed as a pre-treatment to a dyeing or relaxing process and/or a permanent-waving process so as to cosmetically protect the keratin fibres against these treatments. In other words, this process is performed to preserve the cosmetic properties of the keratin fibres before a cosmetic treatment process as described previously.

In particular, the treatment process is performed as a post-treatment to a bleaching, artificial dyeing or relaxing process and/or a permanent-waving process so as to repair the said fibres.

The process according to the invention may be performed during a cosmetic treatment process so as to repair the said fibres.

In particular, the treatment process according to the invention may be performed on damaged keratin fibres.

In other words, the treatment process according to the invention is preferably performed on sensitized keratin fibres, such as bleached, dyed, relaxed or permanent-waved fibres.

In particular, the treatment process may be performed before a bleaching, dyeing or relaxing process and/or a permanent-waving process on keratin fibres.

As a variant, the treatment process may be performed during and/or after a cosmetic treatment process not leading to the artificial dyeing of keratin fibres, in particular:
(a) during and/or after a dyeing process, a process of permanent-waving or a process of relaxing keratin fibres, and
(b) after a process of bleaching keratin fibres.

According to one embodiment, the treatment process according to the invention is performed after a process of bleaching the keratin fibres.

The examples that follow are given as illustrations of the present invention. The amounts indicated in the examples are expressed as weight percentages.

### Oxidized Polysaccharide Tested (Compound 1)

Compound 1 was prepared by oxidation of inulin sold under the name Inutec N25 by the company Orafti, by performing a reactive extrusion process as described in the article “Water-soluble oxidized starches by peroxide reactive extrusion” by R. E. Wing and J. L. Willett, Industrial Crops and Products 7, 1997, pages 45-52. A BC21 co-rotating twin-screw extruder sold by the company Clextral was used, and aqueous hydrogen peroxide solution was used as oxidizing agent.

Compound 1: oxidized inulin obtained by reactive extrusion of a mixture of 78% by weight of inulin and 1.57% by weight of aqueous hydrogen peroxide solution; the spontaneous pH after reactive extrusion is 3.8. Compound 1 thus obtained has a carbonyl content of 1.23% (w/w) and a carboxyl content of 0.17% (w/w).

### Compositions Prepared

| Compositions | A | B | C | X |
|---|---|---|---|---|
| Compound 1 | 1 | 1 | | |
| 3-Aminopropyltriethoxysilane | 1 | | 1 | |
| Water | qs 100 | qs 100 | qs 100 | qs 100 |

The composition to be evaluated is applied at a rate of 10 g of composition per gram of locks. Each composition evaluated is applied on three locks.

Application Process 1:

Locks of hair that was greatly sensitized by bleaching (SA 45%) were used. Composition B (containing 1% of compound 1) was applied to locks of hair and then left on for 15 minutes at 40° C.

The locks were dried manually and composition C (containing the addition of 1% of 3-aminopropyltriethoxysilane in water) was applied and left on for 5 minutes at 40° C., and the locks were then dried manually again.

The locks were then dried under a hood for 15 minutes at 60° C.

The locks were combed before applying a straightening iron at a temperature of 180° C. by performing five continuous passes through the locks for 5 seconds.

For comparative purposes, the same protocol was also performed on locks with, on the one hand, composition B (containing 1% of compound 1) and, on the other hand, composition C (containing the addition of 1% of 3-aminopropyltriethoxysilane to the water). A control lock treated with water, with or without application of the straightening iron was also prepared.

To evaluate the durable (persistent) nature of the cosmetic properties of the locks of hair, they were then washed with one shampoo according to the following protocol:

The treated locks were washed with an aqueous solution containing 15% by weight of sodium lauryl ether sulfate at a rate of 0.4 g of shampoo per gram of hair, at a temperature of 38° C.

Moisten the lock for 5 seconds with water. Apply the shampoo, massaging the lock from the root to the end for 15 seconds. Rinse with water for 10 seconds. Dry manually. Dry the locks for 10 minutes per gram of hair at 60° C. with a hairdryer.

The cosmetic properties of the locks after shampooing was then evaluated, especially the cosmetic feel, the manageability and the ease of combing of the locks, the hair-body effect and the sheen of the hair.

The following results were obtained:

| Type of lock of hair | Cosmetic properties after shampooing |
|---|---|
| Sensitized hair treated with | Coarse feel |
| composition (X) (control) | difficult to comb; dull lock |
| (Lock 1) | |
| Sensitized hair treated with | Less coarse feel |
| composition (X) (control) + heat | Difficult to comb, same level as |
| (Lock 2) | lock 1. Dull lock. |
| Sensitized hair treated with | Slightly manageable lock, easier |
| composition (B) + heat | to comb than lock 1. The hair has |
| (Lock 3) | body. Slightly improved sheen. |
| Sensitized hair treated with | Lock less easy to comb than lock |
| composition (C) + heat | 3 and less shiny, but more man- |
| (Lock 4) | ageable and soft, pleasant cos- |
| | metic feel. |
| | The hair has more body. |
| Sensitized hair treated with | Lock very easy to comb, man- |
| composition (B) and then | ageable and very soft feel, in par- |
| composition (C) + heat | ticular at the ends. The hair has |
| (Lock 5) | the most body and good sheen. |

The locks of hair were then classified as a function of their cosmetic properties (soft, pleasant cosmetic feel, manageability, ease of combing, hair body, sheen) after having performed one shampoo wash.

| | |
|---|---|
| After shampooing | Lock 5 > Lock 3 > Lock 4 > Lock 2 > Lock 1 |

Locks 5 treated via the process according to the invention, and after having undergone 1 shampoo wash or 5 shampoo washes, have better cosmetic properties in terms of soft feel (especially at the hair ends), manageability and ease of combing, and the hair has markedly more body and good sheen. These cosmetic properties thus have good persistence on shampooing.

In addition, the colour of the treated hair is not modified.

Application Process 2:

Locks of hair that were greatly sensitized by bleaching (SA 45%) were used.

Composition A (containing 1% of compound 1 and addition of 1% of 3aminopropyltriethoxysilane to the water) was applied to locks of hair and then left on for 30 minutes at 40° C.

The locks were dried manually and then dried under a hood for 15 minutes at 60° C.

The locks were combed before applying a straightening iron at a temperature of 180° C. by performing five continuous passes through the locks for 5 seconds.

For comparative purposes, the same protocol was also performed on locks with, on the one hand, composition B (containing 1% of compound 1) and, on the other hand, composition C (containing the addition of 1% of 3-aminopropyltriethoxysilane to the water). A control lock treated with water, with or without application of the straightening iron was also prepared.

To evaluate the durable (persistent) nature of the cosmetic properties of the locks of hair, they were then washed with a shampoo according to the protocol described previously.

The following results were obtained:

| Type of lock of hair | Cosmetic properties after shampooing |
|---|---|
| Sensitized hair treated | Coarse feel |
| with composition (X) (control) | Difficult to comb; dull lock |
| (Lock 11) | |
| Sensitized hair treated with | Less coarse feel |
| composition (X) (control) + heat | Difficult to comb, same level as |
| (Lock 12) | lock 11. Dull lock |
| Sensitized hair treated with | Slightly manageable lock easier to |
| composition (B) + heat | comb than lock 11. The hair has |
| (Lock 13) | body. Sheen slightly improved. |
| Sensitized hair treated with | Lock less easy to comb than lock |
| composition (C) + heat | 13 and less shiny, but more man- |
| (Lock 14) | ageable and soft, pleasant cosmet- |
| | ic feel. The hair has more body. |
| Sensitized hair treated with | Lock very easy to comb, manage- |
| composition (A) + heat | able, and with a softer feel. The |
| (Lock 15) | hair has more body and good |
| | sheen. |

The locks of hair were then classified as a function of their cosmetic properties (soft, pleasant cosmetic feel, manageability, ease of combing and resistance, hair body, sheen) after having been shampooed.

| | |
|---|---|
| After shampooing | Lock 15 > Lock 13 > Lock 14 > Lock 12 > Lock 11 |

Lock 15 treated via the process according to the invention, and after having undergone a shampoo wash has better cosmetic properties in terms of soft feel, manageability and ease of combing, and the hair has more body and good sheen. These cosmetic properties thus have good persistence on shampooing. In addition, the colour of the treated hair is not modified.

The process was also applied to type IV natural curly hair.

The following results were obtained:

| Type of lock of hair | Cosmetic properties after shampooing |
|---|---|
| Type IV natural hair treated with | Dry, coarse feel. |
| composition (X) (control) | Difficult to comb. |
| (Lock 11′) | |
| Type IV natural hair treated with | Less coarse feel. |
| composition (X) (control) + heat | Difficult to comb, same level as |
| (Lock 12′) | lock 11′. |
| Type IV natural hair treated with | Slightly manageable lock, easier |
| composition (B) + heat | to comb than lock 11′. The hair |
| (Lock 13′) | has body. Slightly improved |
| | sheen. |
| Type IV natural hair treated with | Lock less easy to comb than lock |
| composition (C) + heat | 13′ and less shiny, but more |
| (Lock 14′) | manageable and soft, pleasant |
| | cosmetic feel. The hair has more |
| | body. |
| Type IV natural hair treated with | Lock very easy to comb, man- |
| composition (A) + heat | ageable and softer feeling. The |
| (Lock 15′) | hair has more body and good |
| | sheen. It also has better shaping. |

| | |
|---|---|
| After shampooing | Lock 15′ > Lock 13′ > Lock 14′ > Lock 12′ > Lock 11′ |

The locks 15′ treated via the process according to the invention, and after having been shampooed, have better cosmetic properties in terms of a soft feel, manageability and ease of combing, and the hair has more body, good sheen and good shaping. These cosmetic properties are thus persistent on shampooing. In addition, the colour of the treated hair is not modified.

Application Process 3:

Locks of hair that were greatly sensitized by bleaching (SA 45%) were used.

Composition (B) (containing 1% of compound 1) was applied to locks of hair and then left on for 15 minutes at 40° C.

The locks were dried manually and then dried under a hood for 15 minutes at 60° C.

The locks were combed before applying a straightening iron at a temperature of 180° C. by making five continuous passes through the locks for 5 seconds.

Composition C (containing the addition of 1% of 3-aminopropyltriethoxysilane to the water) was then applied and left on for 15 minutes at 40° C., and the locks were then dried manually again.

The locks were then dried under a hood for 15 minutes at 60° C.

The locks were combed before applying a straightening iron at a temperature of 180° C. by making five continuous passes through the locks for 5 seconds.

To evaluate the lasting (persistent) nature of the cosmetic properties of the locks of hair, they were then washed with a shampoo according to the protocol described previously.

The following results were obtained:

| Type of lock of hair | Cosmetic properties after shampooing |
|---|---|
| Sensitized hair treated with | Coarse feel |
| composition (X) (control) | Difficult to comb; dull lock |
| (Lock 21) | |
| Sensitized hair treated with | Less coarse feel |
| composition (X) (control) + heat | Difficult to comb, same level as |
| (Lock 22) | lock 21. Dull lock. |
| Sensitized hair treated with | Slightly manageable lock, easier |
| composition (B) + heat | to comb than lock 21. The hair |
| (Lock 23) | has body. Slightly improved |
| | sheen. |
| Sensitized hair treated with | Lock less easy to comb than |
| composition (C) + heat | Lock 23 and less shiny, but more |
| (Lock 24) | manageable and soft, pleasant |
| | cosmetic feel. The hair has more |
| | body. |
| Sensitized hair treated with | More manageable lock and the |
| composition (B) and then | hair has more body. |
| composition (C) + heat | |
| (Lock 25) | |

The locks of hair were then classified as a function of their cosmetic properties (manageability and body of the hair) after having been shampooed.

| | |
|---|---|
| After shampooing | Lock 25 > Lock 23 > Lock 24 > Lock 22 > Lock 21 |

The locks 25 treated via the process according to the invention, and after having been shampooed, have better cosmetic properties in terms of manageability and the hair has markedly more body. These cosmetic properties thus have good persistence on shampooing.

In addition, the colour of the treated hair is not modified.

1.-27. (canceled)

28. A process for treating keratin fibers, the process comprising:
(i) applying to the keratin fibers at least one oxidized polysaccharide,
(ii) heating the keratin fibers at a temperature of at least about 100° C.,
(iii) applying to the keratin fibers an aqueous cosmetic composition comprising at least one silane of formula (II):
R₁Si(OR₂)_{z}(R₃)ₓ (II)
wherein:
R₁ is a linear or branched, saturated or unsaturated, cyclic or acyclic C₁-C₆ hydrocarbon-based chain substituted with a group chosen from the following groups:
amine NH₂ or NHR wherein R=C₁-C₄ alkyl,
an aryl or aryloxy group substituted with an amino group or with a C₁-C₄ am inoalkyl group;
wherein R₁ is optionally interrupted in its chain with a heteroatom, O, S, NH, or a carbonyl group (CO), R₁ being linked to the silicon atom directly via a carbon atom,
R₂ and R₃, which may be identical or different, are chosen from linear or branched alkyl groups comprising from 1 to 6 carbon atoms,
z is chosen from an integer ranging from 1 to 3, and
x is chosen from an integer ranging from 0 to 2,
with z+x=3.

29. The process according to claim 28, wherein the at least one oxidized polysaccharide is anionic or nonionic.

30. The process according to claim 28, wherein the at least one oxidized polysaccharide comprises at least one aldehyde group and optionally at least one anionic group, carboxyl group, and/or carboxylate group.

31. The process according to claim 28, wherein the at least one oxidized polysaccharide is chosen from those of formula (I):
P—(CHO)m(COOX)n (I)
wherein:
P represents a polysaccharide chain,
X is chosen from a hydrogen atom, ions derived from an alkali metal or an alkaline-earth metal, sodium ion, potassium ion, ammonia, organic amines, monoethanolamine, diethanolamine, triethanolamine, 3-amino-1,2-propanediol, basic amino acids, lysine, arginine, sarcosine, ornithine, or citrulline,
m+n is greater than or equal to 1,
m is such that the degree of substitution of the polysaccharide with at least one aldehyde group (DS(CHO)) ranges from about 0.001 to about 2, and
n is such that the degree of substitution of the polysaccharide with at least one carboxylic group (DS(COOX)) ranges from about 0 to about 2.

32. The process according to claim 31, wherein the polysaccharide chain is chosen from celluloses, hydroxyethylcelluloses, hydroxypropylcelluloses, carboxymethylcelluloses, starches, guar gums, inulins, xanthan gums, pullulan gums, agar-agar gums, carrageenan gums, gellan gums, gum arabics, tragacanth gums, xylans and derivatives thereof, cellobiose, maltodextrin, scleroglucan, chitosan, ulvan, fucoidan, alginate, pectin, heparin, or hyaluronic acid.

33. The process according to claim 28, wherein in the silane of formula (II):
R₁ is a saturated linear C₁-C₆ hydrocarbon-based chain substituted with an amine group NH₂,
R₂ is chosen from an alkyl group comprising from 1 to 4 carbon atoms, and
R₃ is chosen from an alkyl group comprising from 1 to 4 carbon atoms.

34. The process according to claim 28, wherein in the silane of formula (II), z is equal to 3.

35. The process according to claim 28, wherein the silane of formula (II) is chosen from 3-aminopropyltriethoxysilane, 3-aminoethyltriethoxysilane, 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane, or N-(2-aminoethylaminomethyl)phenethyltrimethoxysilane.

36. The process according to claim 28, wherein applying the oxidized polysaccharide comprises applying a cosmetic composition comprising the oxidized polysaccharide in an amount ranging from about 0.05% to about 15% by weight, relative to the total weight of the cosmetic composition.

37. The process according to claim 28, wherein the silane of formula (II) is added to the aqueous composition in an amount ranging from about 0.1% to about 15% by weight, relative to the total weight of the cosmetic composition.

38. The process according to claim 28, wherein the heating step is performed at a temperature ranging from about 100° C. to about 250° C.

39. The process according to claim 28, wherein the steps are performed, in a sequence chosen from the following:
applying the at least one oxidized polysaccharide or a first cosmetic composition comprising the at least one oxidized polysaccharide, then applying the aqueous cosmetic composition comprising the at least one silane of formula (II), and then heating the keratin fibers;
simultaneously, applying the at least one oxidized polysaccharide or a first cosmetic composition comprising the at least one oxidized polysaccharide, and the aqueous cosmetic composition comprising the at least one silane of formula (II), and then heating the keratin fibers;
applying an aqueous cosmetic composition comprising both the at least one oxidized polysaccharide and the at least one silane of formula (II), and then heating the keratin fibers;
applying the at least one oxidized polysaccharide or a first cosmetic composition containing the at least one oxidized polysaccharide, then heating the keratin fibers, then applying the aqueous cosmetic composition comprising the at least one silane of formula (II), and then optionally heating the keratin fibers an additional time.

40. The process according to claim 28, wherein the process further comprises drying the keratin fibers after the application of the at least one oxidized polysaccharide or of a cosmetic composition containing the same and/or of the aqueous cosmetic composition comprising the added silane (II), and before heating the keratin fibers, the drying step being performed at a temperature ranging from about 20° C. to about 70° C.

41. The process according to claim 28, wherein after application to the keratin fibers of the at least one oxidized polysaccharide or of a cosmetic composition containing the same and/or of the aqueous cosmetic composition comprising the at least one silane of formula (II), and before heating the keratin fibers, the at least one oxidized polysaccharide or the composition containing the same and/or the aqueous cosmetic composition comprising the at least one silane of formula (II) is left on the keratin fibers for a time ranging from about 1 to about 60 minutes.

42. The process according to claim 28, wherein the heating step is performed with a straightening iron.

43. The process according to claim 28, wherein the heating step is performed by applying a straightening iron to the keratin fibers in a substantially continuous movement from the root to the end of the keratin fibers, in at least one pass.

44. The process according to claim 28, wherein the process is performed on damaged keratin fibers and/or hair.

45. The process according to claim 28, wherein either or both of the at least one oxidized polysaccharide and/or the at least one silane of formula (II) is present in a cosmetic composition comprising a physiologically acceptable aqueous medium.

46. A cosmetic composition comprising, in a physiologically acceptable aqueous medium, at least one oxidized polysaccharide chosen from oxidized inulin, and at least one silane of formula (II):
R₁Si(OR₂)_{z}(R₃)ₓ (II)
wherein:
R₁ is a linear or branched, saturated or unsaturated, cyclic or acyclic C₁-C₆ hydrocarbon-based chain substituted with a group chosen from the following groups:
amine NH₂ or NHR wherein R=C₁-C₄ alkyl,
an aryl or aryloxy group substituted with an amino group or with a C₁-C₄ am inoalkyl group;
wherein R₁ is optionally interrupted in its chain with a heteroatom, O, S, NH, or a carbonyl group (CO), R₁ being linked to the silicon atom directly via a carbon atom,
R₂ and R₃, which may be identical or different, are chosen from a linear or branched alkyl group comprising from 1 to 6 carbon atoms,
z is chosen from an integer ranging from 1 to 3, and
x is chosen from an integer ranging from 0 to 2,
with z+x=3.

47. A kit comprising:
a cosmetic composition packaged in a packaging assembly, the cosmetic composition comprising at least one oxidized polysaccharide and at least one silane; or
a first cosmetic composition comprising at least one oxidized polysaccharide and a second cosmetic composition comprising at least one silane, wherein the first cosmetic composition and the second cosmetic composition are each packaged in separate packaging assemblies;
wherein the oxidized polysaccharide is oxidized inulin, and
wherein the at least one silane is a silane of formula (II):
R₁Si(OR₂)_{z}(R₃)ₓ (II)
wherein:
R₁ is a linear or branched, saturated or unsaturated, cyclic or acyclic C₁-C₆ hydrocarbon-based chain substituted with a group chosen from the following groups:
amine NH₂ or NHR wherein R=C₁-C₄ alkyl,
an aryl or aryloxy group substituted with an amino group or with a C₁-C₄ am inoalkyl group;
wherein R₁ is optionally interrupted in its chain with a heteroatom, O, S, NH, or a carbonyl group (CO), R₁ being linked to the silicon atom directly via a carbon atom,
R₂ and R₃, which may be identical or different, are chosen from a linear or branched alkyl group comprising from 1 to 6 carbon atoms,
z is chosen from an integer ranging from 1 to 3, and
x is chosen from an integer ranging from 0 to 2,
with z+x=3; and
wherein the kit optionally comprises a device for heating keratin fibers at a temperature of at least about 100° C.
